(19) United States
(12) Patent Application Publication
(10) Pub. No.: US 2013/0123703 A1
(43) Pub. Date: May 16, 2013

(54) PULSE INFUSION DEVICE SYSTEM AND METHOD

(71) Applicant: Ofer Shay, Kfar Vradim (IL)

(72) Inventor: Ofer Shay, Kfar Vradim (IL)

(21) Appl. No.: 13/811,348

(22) PCT Filed: Jul. 21, 2011
(86) PCT No.: PCT/IL11/00591

§ 371 Date: Jan. 21, 2013

Related U.S. Application Data
(60) Provisional application No. 61/382,797, filed on Sep. 14, 2010.
(60) Provisional application No. 61/366,688, filed on Jul. 22, 2010.

(51) Int. Cl. A61M 5/145

(52) U.S. Cl. CPC A61M 5/1452
USPC 604/153

(57) ABSTRACT

A device, system and method are disclosed for providing infused medication in a continuous pulse flow at a defined volume and frequency while maintaining a stable and accurate average flow rate, in order to provide improved nerve bathing and thus continuous pain blockage. The system may comprise an external reservoir, such as an infusion bag, and a pulsed flow generation device for generating a controlled pulse of fluid received from the external reservoir and retained in an internal reservoir such as a disposable syringe. One method according to an embodiment of the present invention may comprise receiving a fluid medication, such as an anesthetic substance, from an external reservoir, containing the fluid in an internal reservoir, and when the volume of fluid reaches a certain predefined value, releasing at least one pulse of fluid to create nerve bathing at a treated area.

### FIELD OF INVENTION

The present invention relates to the administration of liquid medicines. More particularly there is disclosed a pulse infusion pump which is programmable to suit the volume and frequency as directed by the doctor in charge of the patient or by the patient him/herself in pain control applications.

### BACKGROUND OF THE INVENTION

Since the early 90's the use of infusion pumps to continuously administer anesthetics has become common practice for achieving long-term regional anesthesia. These pumps are either electro-mechanical pumps or mechanical pumps. Most pumps are designed to be ambulatory, carried by the patient in a pouch or similar holder. Some types of pump are suitable for PCA (patient control analgesia) whereby the patient can add additional medication bolus to the basal flow to address severe pain.

Currently there are two main clinical procedures that are used for continuous long-term post operative regional/local anesthesia, both are subcutaneous/intramuscular: Surgical Site Infiltration wherein the medication is introduced into or nearby the surgical incision by use of a catheter with a long fenestrated segment inserted into the patient tissue. In the second procedure, Continuous Peripheral Nerve Block (CPNB) medication is introduced proximate to the nerve that controls the limb that has been operated. When CPNB administration is performed, an efficient pain block is achieved when at one location the nerve is saturated 360° by the medication. Therefore maintaining sufficient nerve bathing is essential to gain continuous pain blockage. For example, such sufficient nerve bathing is achieved when a nerve block is performed by manual injection, typically performed prior to surgery. One of the main objectives of the present innovation is to continuously maintain sufficient nerve bathing through implementing an innovative infusion strategy for CPNB and thereby gain an improved post operative pain therapy.

### SUMMARY OF THE INVENTION

The device of the invention provides infused medication in a continuous pulse flow at a defined volume and frequency while maintaining a stable and accurate average flow rate. The device is particularly useful for large volume pulses at low frequency.

Where P=volume of pulse

The average flow rate Fr=ΣP/T (wherein ΣP is the total volume of pulses and T is time);

M=P*Fr=pulse flow-rate multiply.

Depending on the anatomy of the specific nerve, nerve bathing is affected by the setup of these parameters. Therefore, it is clinically important that these parameters can be controlled and set by the medical team.

As the device is mainly intended to be used for continuous regional anesthesia that is performed through CPNB, the high M value results in improved nerve bathing leading to improved anesthesia.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B are schematic illustrations of an electro-mechanical pulse infusion system according to one embodiment of the present invention in a pre-pulse position and in a post-pulse position;

FIGS. 2, 3, 4, 5, 6A, 6B, 7A and 7B are schematic illustrations of additional embodiments of a mechanical and electro-mechanical pulse infusion system according to the present invention;

FIG. 8 is a schematic illustration of a mechanical pulse flow generation device according to one embodiment of the present invention; and

FIG. 9 is a flowchart of a method for converting a constant flow into a pulse flow according to an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

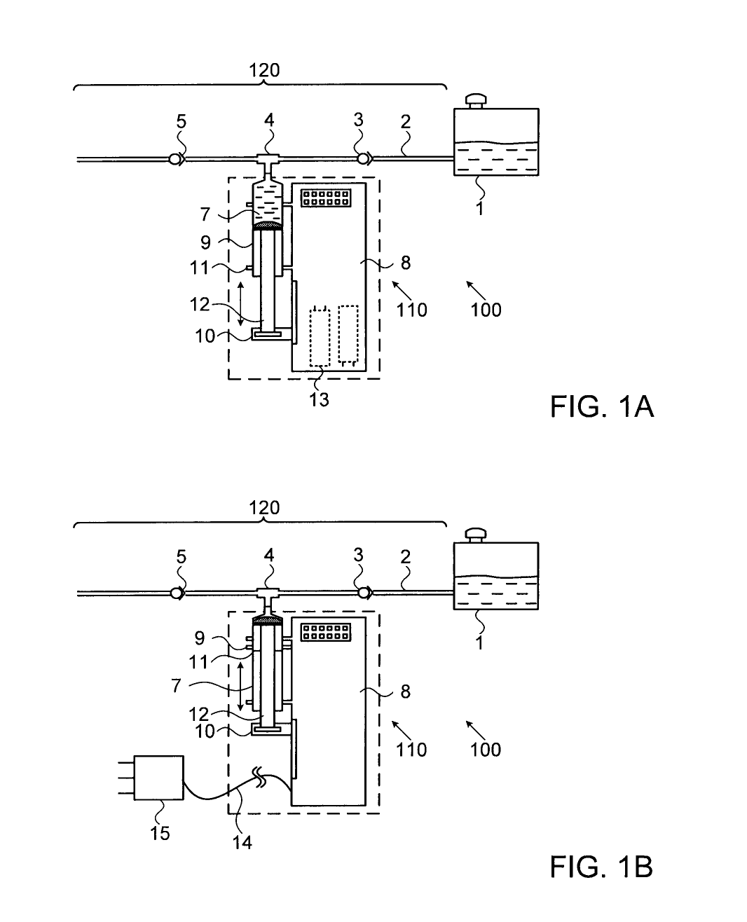

System 100, which is illustrated in FIGS. 1A and 1B, is a stand-alone electro-mechanical infusion system that creates pulsed flow having a high M value. According to some embodiments of the present invention system 100 may allow a user to set the volume of the pulse, the frequency of the pulses and/or the pulse velocity.

Where P=volume of pulse

The average flow rate Fr=ΣP/T (wherein ΣP is the total volume of pulses and T is time);

M=P*Fr=pulse flow-rate multiply. Equation 1:

According to one embodiment of the present invention, system 100 is connected to an external reservoir 1 which may be a fluid medication reservoir; solid, semi-solid container or a bag, a tubing system 120 and a pulsed flow generation device 110. According to some embodiments of the present invention, tubing system 120 may be a disposable tubing system. According to other or additional embodiments pulsed flow generation device 110 may be programmable by a user such as a medical team and/or a patient. According to yet another embodiment of the present invention, pulsed flow generation device 110 may be pre-set.

Pulsed flow generation device 110 may comprise an internal pump reservoir, such as syringe 7, a piston 12 and a pulse actuation apparatus 8. During operation syringe 7 is filled and emptied during each cycle.

According to one embodiment of the present invention, syringe 7 is filled using energy provided by the flow from external reservoir 1. It would be appreciated by those skilled in the art that other mechanisms may be used for filling syringe 7 with fluid received from external reservoir 1.

According to one embodiment of the present invention, pulsed flow generation device 110 may be operated electromechanically, through an electric motor or solenoid (not shown) which may be controlled by an electronic controller (not shown) in actuation apparatus 8. The electronic controller may be programmable or preprogrammed to allow adapting the pulses frequency, the volume of each pulse of fluid and other parameters in order to tailor these parameters to the needs of each patient.

According to yet another embodiment of the present invention, device 110 may comprise a plurality of controllers (not shown), each of said controllers may control a different parameter of Equation 1 above. For instance, actuation apparatus 8 may comprise a controller for controlling the pulses frequency (not shown). According to another embodiment of the present invention, actuation apparatus 8 may comprise another or an additional controller such as a pulsed flow volume controller. Alternatively or additionally, actuation apparatus 8 may comprise a flow velocity controller. It would be appreciated by those skilled in the art that other controllers, optionally of other parameters, may be used.

Pulsed flow generation device 110 may pump a defined volume of fluid received from external reservoir 1 to an internal pump reservoir, such as syringe 7. Piston 12 may then pump out that defined volume, entirely or partially, into a catheter (not shown) placed in the body of the patient. These pumping operations may be performed continuously at a selected frequency.

According to one embodiment of the invention both syringe 7 and piston 12 may be parts of a disposable syringe set. Device operation parameters can be preset during manufacturing (pre-programmed) or, in a programmable version, the medical team may have the option to select and set the operational parameters of the device during the course of the therapy and to permanently lock them when needed.

Advantageously the device may be an ambulatory type powered by batteries 13. However a stationary device can be used where the patient is unlikely to be moved. Energy may then be supplied through a cord 14 connected to the building electric supply via a transformer-rectifier 15.

FIG. 1A represents an electromechanical pulsed flow generation device 110. Tubing system 120 compromise tube 2 that may be connected at one end to external reservoir 1 by use of a standard fitting and on the other end to check valve 3. A connector, such as a T shape connector 4, is positioned between said check valve and pressure activated check valve 5. Outlet port 6 is positioned after said pressure activated check valve. Outlet port 6 may have standard fitting to be connected to an NB catheter placed in the patient body or any other fluid insertion apparatus known in the art. The remaining branch of T connector 4 opens into variable volume container such as a standard disposable syringe 7. It would be appreciated by those skilled in the art that actuation apparatus 8 of device 110 may be disposable or reusable, while tubing system 120 and external reservoir 1 are usually disposable components.

Syringe 7 may be connected to electromechanical programmable actuation apparatus 8 by mounting the syringe barrel 11 onto a holder 9 and the piston rod 12 to the pull lever 10.

Check valve 3 prevents back-flow of fluids from connector 4 to external reservoir 1. Pressure-activated check valve 5 prevents gravity flow from reservoir 1 to exit port 6.

Pull lever 10 of actuation apparatus 8 may move linearly only along one axis of piston 12 (in the direction of the double-headed arrow indicated in FIGS. 1A and 1B) so that when pull lever 10 moves in a first direction, the internal volume of syringe 7 increases and when pull lever 10 moves in a second direction the volume of internal volume of syringe 7 decreases.

Movement in the first direction of the pull lever 10, driven by the actuation apparatus 8, draws the piston 12 in the same first direction, creating a vacuum in the cylinder of syringe which serves as internal intermittent reservoir 7. As a result fluid is drawn from reservoir 1 into syringe 7.

Movement of pull lever 10 in said second direction applies pressure on the fluid in syringe 7 that pumps out the medication from said syringe 7 to the patient through pressure-activated check valve 5 and through outlet port 6.

Electronic programmable means of actuation apparatus 8 enables to determine the volume that is pumped into syringe 7 every and each movement cycle of pull lever 10 in the first direction and the volume that is pumped out of syringe 7 every and each movement of pull lever 10 in the second direction. Frequency of pull lever 10 movement may also be pre-set and controlled. Similarly, the speed movement pull lever 10 may also be pre-set and controlled.

According to some embodiments of the present invention, actuation apparatus 8 may be equipped with electronic means to store and analyze the infusion data and to sound an alarm when data received and recorded is outside pre-defined limits. For example, when the total pulsed flow volume is beyond a predefined maximum dosage.

FIG. 1B shows the electromechanical pulse infusion system 100, presenting the system in a situation where the pull lever 10 has moved in the second direction to its extremity, i.e. pumping out the fluids within syringe 7. According to the embodiment illustrated in FIG. 1B, device 110 may be arranged to receive power from a wall socket, using a transformer-rectifier 15 and a cable 14.

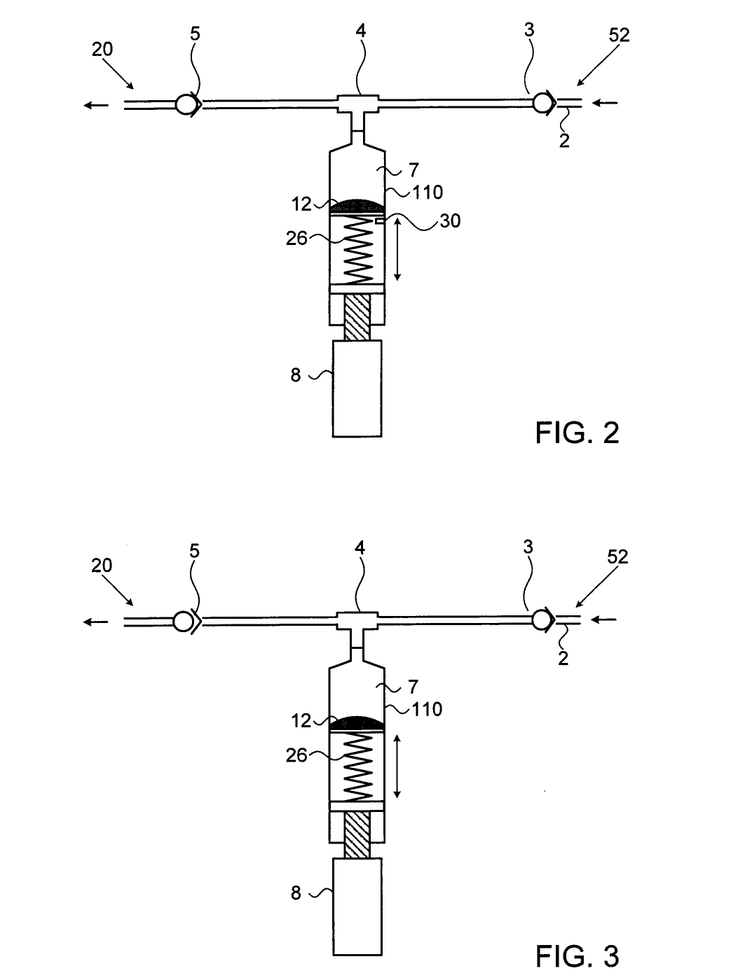

Reference is now made to FIG. 2 which is a schematic drawing of another electromechanical embodiment of the present invention. As may be seen in FIG. 2, tubing 2 is connected to an inlet port 52 through an optional one-way valve 3. A connector such as a T shape connector 4 leads to a pressure-activated check valve 5 and an exit port 20.

Pulse flow generation device 110 is also connected to the ‘T’ connector 4. Pulsed flow generation device 110 is equipped with a piston 12, an optional spring 26, an electric actuation apparatus 8 and a sensor (proximity switch) 30. syringe 7 is filled and discharges through connector 4.

A fluid, such as fluid medicament, may flow from an infusion pump (not shown) through inlet port 52, and through valve 3. The fluid flowing into tube 2 between check valves 3 and 5 may cause pressure build-up and push piston 12 in the first direction to increase the volume of fluid that may be contained in syringe 7. When the volume of fluid within syringe 7 reaches a predefined volume, actuation apparatus 8 causes piston 12 to start moving in a second direction to pump out the fluid contained in syringe 7. When fluid is pumped out from syringe 7 into tube 2, pressure in tube 2 increases until pressure activated check valve 5 is opened, and a pulse of fluid may flow through the pressure-activated check valve 5 and may exit into a patient's body through port 20.

According to one embodiment of the present invention, as piston 12 reaches the vicinity of proximity switch 30 an electric signal causes actuation apparatus 8 to move in a second direction and applies an additional force on compression spring 26. Spring 26 in turn pushes liquid out of device 110 forcing valve 5 to open and release a pulse of fluid medication. Spring 26 acts as a buffer between the fast actuation apparatus 8 and the slower movement of the piston 12. According to yet another embodiment of the present invention, actuation apparatus 8 retracts to its original position after a preset delay, typically between 1 and 3 seconds. The reduced fluid pressure in syringe 7 allows new fluid therein thus starting a new cycle.

It would be appreciated by those skilled in the art that spring 26 may not be required and other buffer mechanisms may be used. It would be further realized that a buffer may not be required at all.

Means are provided to change the position of sensor or proximity switch 30, thus adjusting the pulsed fluid volume. Other means for adjusting the volume of fluid released in each pulse may be used.

In an alternative embodiment sensor 30 is a component which continuously monitors piston 12 position and transmits signals to a programmable controller (PEC) (not seen). The PEC is easily set to a desired fluid volume per pulse, and additionally any desired time delay can be programmed therein.

Referring now to FIG. 3 there is seen an embodiment of pulse flow generation device 110 which is identical to that seen in FIG. 2 except that no sensor (proximity switch) is provided. A PEC (not shown) controls the actuation apparatus 8, generating an electric signal according to a time interval set by the medical team. The signal connects power to the actuation apparatus 8 to move in a second direction to pump out fluid from syringe 7 and the pulse is generated exactly as described with reference to FIG. 2. The time interval set in the PEC may be easily changed, and thus different pulsed volumes can be ejected while using the same basic flow rate.

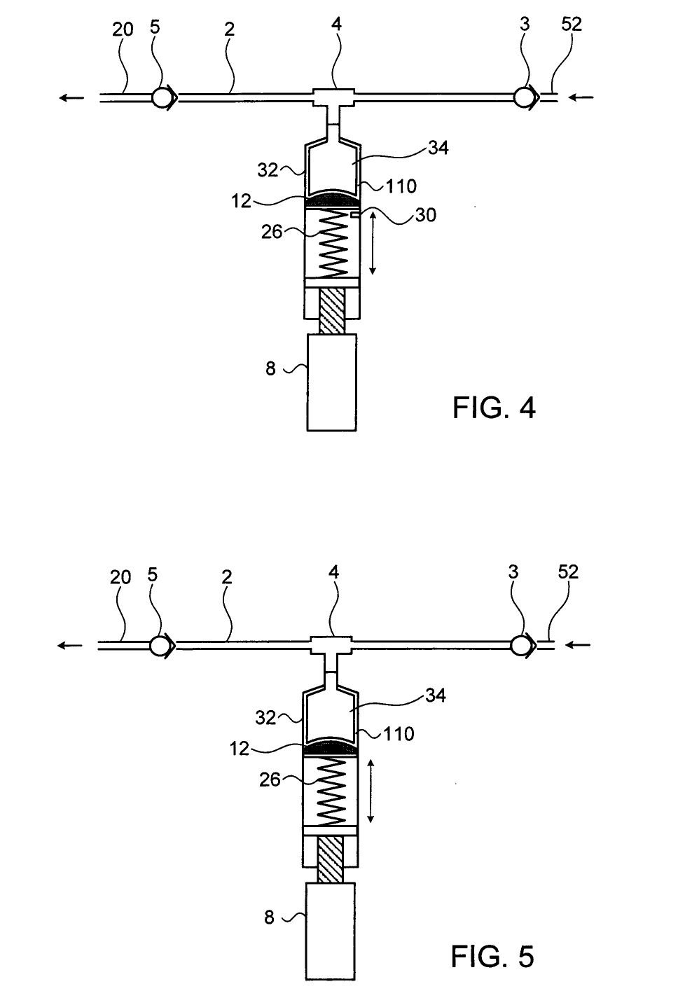

Turning now to FIG. 4, there is seen an embodiment provided with a syringe 7 having an internal container 34 made of an elastic material, for example of silicone rubber positioned inside a rigid container 32. Internal container 34 has a controlled volume and is beneficial in preventing any leak of a fluid into the pump mechanism. Furthermore, Internal container 34 reduces the area of contact between the fluid and parts of the pump. In all other respects the present embodiment is identical to the embodiment described with reference to FIG. 2.

With regard to FIG. 5, there is seen an embodiment which is the same as that shown in FIG. 4, except that a PEC (not shown) comprised within actuation apparatus 8 creates an electric signal according to a time interval set by the user. Therefore switch or sensor 30 seen in FIG. 4 may not be required.

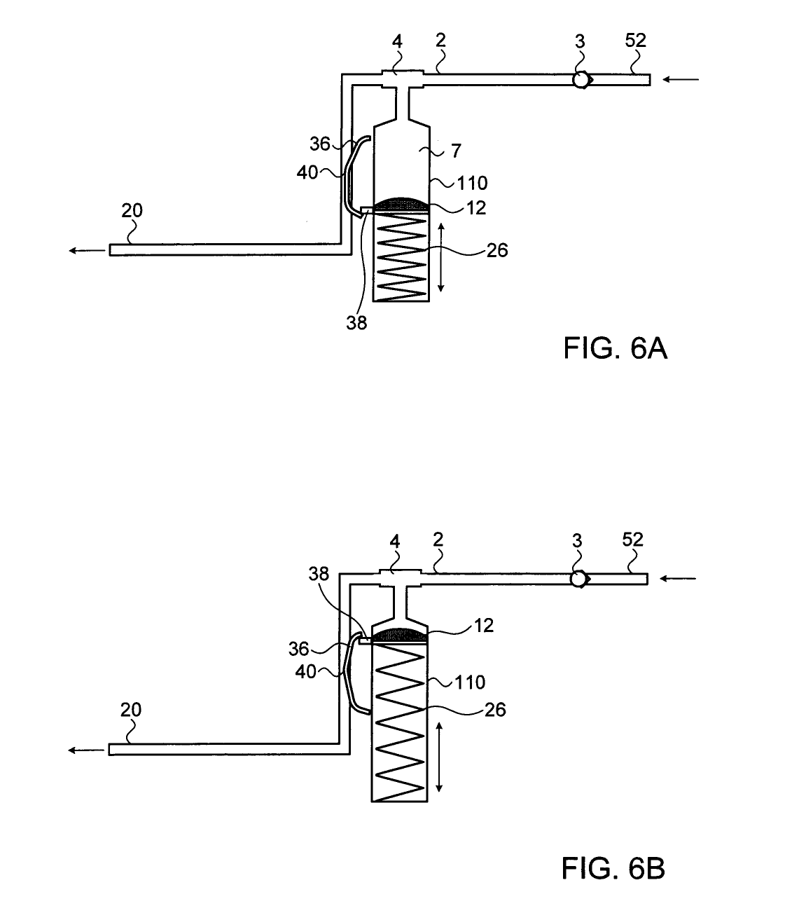

FIGS. 6a and 6b illustrate a mechanical pulse device, so there is no electric actuation apparatus 8 as was seen in previous embodiments.

Tubing 2 is connected to an inlet port 52 through an optional one-way valve 3. A connector such as T shaped connector 4 leads to a pressure-activated check valve 40 and an exit port 20.

Pulsed flow generation device 110 is also connected to the ‘T’ connector 4. Pulsed flow generation device 110 may be equipped with a piston 12, a spring 26, and a projection 38.

The normally closed valve 40 thus prevents fluid discharge through outlet port 20, wherefore incoming fluid accumulates in syringe 7.

Valve 40 may be actuated by a lever 36 when pushed by projection 38.

A fluid, such as a fluid medicament may flow from an infusion pump (not seen) through inlet port 52. During pressure build up in connector 4 and in the syringe 7 piston 12 moves in a first direction to increase the volume of fluid contained in syringe 7 until projection 38 contacts a part of lever 36, opening valve 40 and forcing a pulse of liquid through port 20.

The reduced fluid pressure in syringe 7 then allows the entry of new fluid into syringe 7 thus starting the next cycle.

Means are provided to change the position of the projection 38 relative to the dimensions of pulse flow generation device 110, thus adjusting the pulse volume. According to another embodiment, two projections, lower and upper may be used instead of projection 38. The lower projection can be adjusted by the medical team member for varying the pulse volume. It would be appreciated that other means for adjusting the pulsed volume may be used.

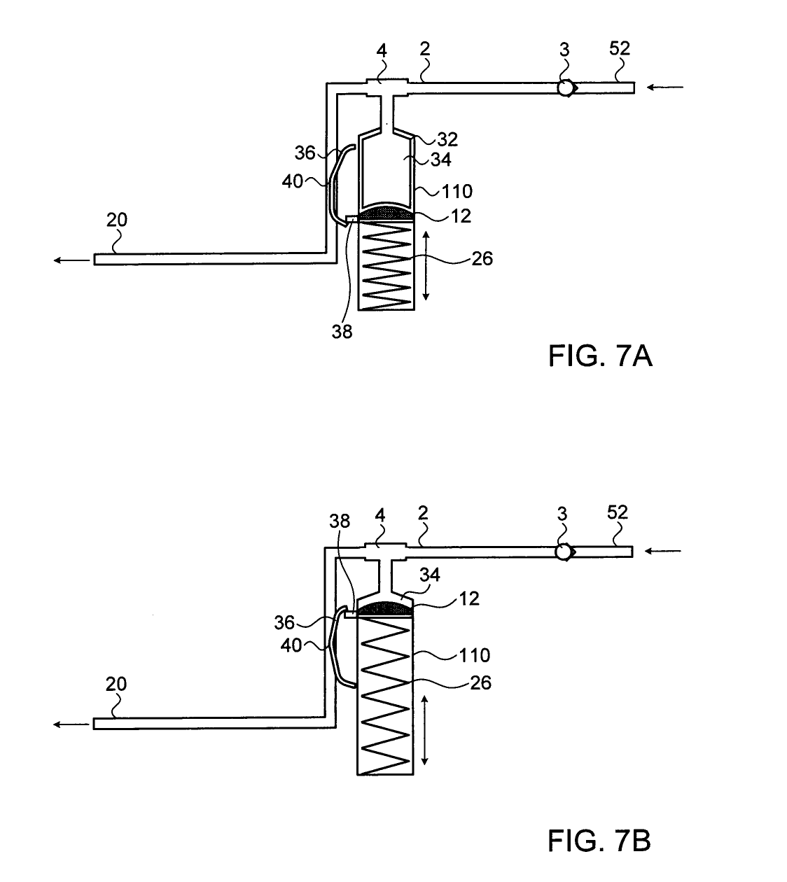

Turning now to FIGS. 7a and 7b there is seen the same embodiment shown in the previous figures, FIGS. 6a and 6b, the only difference being that syringe 7 comprises an internal container made of an elastic material, for example of silicone rubber The advantages of this arrangement have been explained with reference to FIG. 4.

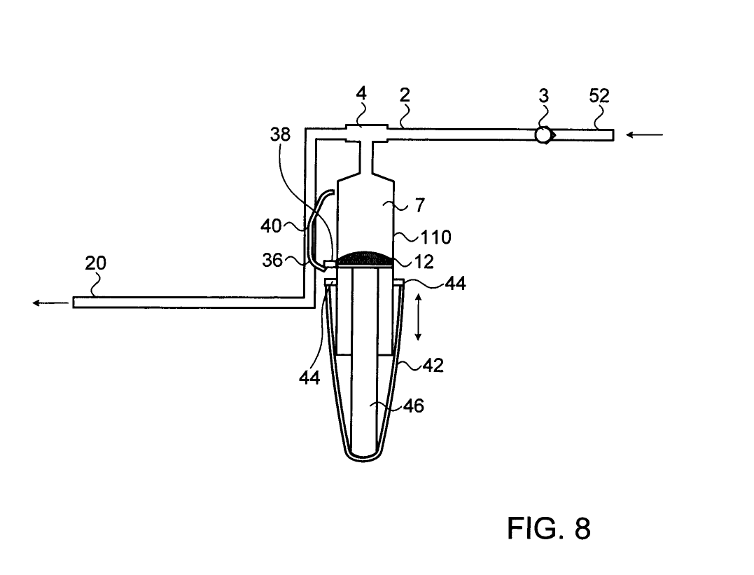

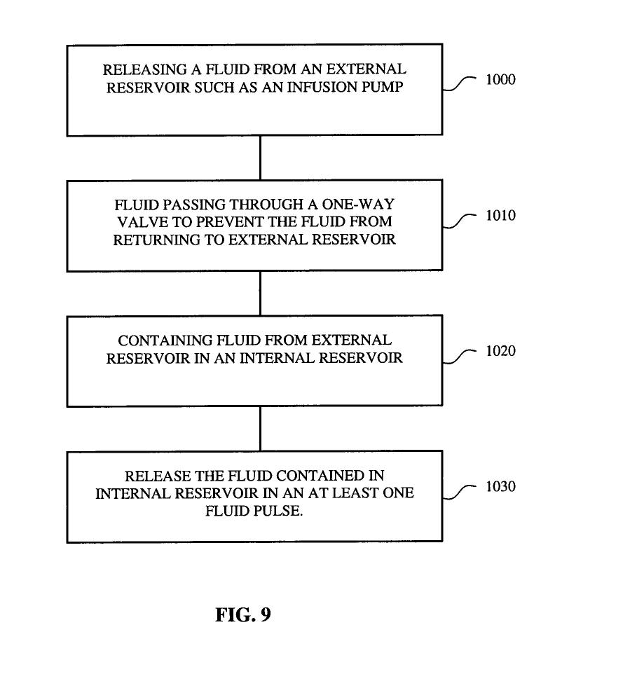

Referring now to FIG. 8, there is seen an arrangement of a mechanical pulse device that is similar to the device seen in FIGS. 6a and 6b. An elastic band 42 is connected to projections 44 while being tensioned over a piston rod 46. The elastic band 42 thus replaces the compression spring 26 seen in previous embodiments, and being external can be easily replaced when necessary.

The pulsed flow generation device 110 can be an integral part of an infusion pump or may be connectable to any infusion pump known in the art.

Reference is now made to FIG. 9 which is a flowchart of a method for converting a constant flow into a pulse flow according to an embodiment of the present invention. The method comprising the following steps:

Releasing a fluid, such as an infusion medicament, from an external reservoir such as an infusion pump [Block 1000]. The fluid may than pass through a one-way valve to prevent the fluid from returning to the external reservoir [Block 1010].

Since the fluid flowing form the external reservoir is prevented from returning to the reservoir by the one-way valve, and cannot pass another valve, such as a pressure operated valve, the fluid enters and contained in an internal reservoir, such as a syringe [Block 1020].

When the volume of fluid in the internal reservoir reaches a predefined value, an actuation apparatus applies pressure on the fluid contained in the reservoir and thus releases the contained fluid in an at least one pulsed flow [Block 1030].

According to one embodiment of the present invention, the volume of fluid contained in the internal reservoir may be released in several consecutive pulses, each pulse having a volume which is relative to the number of pulses. For example, if the reservoir has been filled with 30 ml of fluid medication, it may be released in one pulse of 30 ml, or may be released in 3 consecutive pulses of 10 ml. each.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A pulse infusion system comprising:
an external reservoir adapted to contain infusion fluids;
a tubing system connected at a first end to said external reservoir and at a second end to an insertion unit;
said tubing system further comprises a first check valve proximate to said first end and a second check valve proximate to said second end; and a pulse flow generation device to generate a pulse of infusion fluid;
wherein said pulse flow generation device comprises:
an internal reservoir;
a piston; and
an actuation apparatus connected to said piston.

2. The system of claim 1 wherein said actuation apparatus and said piston connected thereto, are movable in a first direction to increase the volume of said internal reservoir and in to second direction, opposite to said first direction to decrease the volume of said internal reservoir and release a pulse of said infusion fluids.

3. The system of claim 2 wherein said actuation apparatus is selected from a group comprising: an electromechanical actuator a mechanical actuator.

4. (canceled)

5. The system according to claim 3 wherein the volume, the frequency and the velocity of each of said pulses of said infusion fluids may be adjustable.

6. The system according to claim 1 wherein said second check value is a pressure operated check valve.

7. The system according to claim 2 wherein said tubing system further comprises a connector to connect said internal reservoir to said tubing system.

8. A pulse flow generation device for transferring a constant flow of infusion fluids to a pulse flow, said device comprising:
an internal reservoir;
to piston; and
an actuation apparatus connected to said piston.

9. The device according to claim 8 further comprising a sensor, said sensor is connected to said internal reservoir and to said actuation apparatus, to send a signal to said actuation apparatus when the volume of fluid in said reservoir reaches a predefined volume.

10. The device according to claim 8 wherein said internal reservoir is a rigid barrel.

11. The device according to claim 8 wherein said internal reservoir comprises an internal container made of an elastic material.

12. The device according to claim 8 further comprising at least one controller.

13. The device according to claim 12 wherein each of said at least one controllers is selected from a group comprising: a pulse frequency controller, a release velocity controller, and as pulse volume controller.

14. The device according to claim 8 further comprising a spring to provide pressure on said piston to release a pulse of infusion liquid from said reservoir.

15. The device according to claim 8 further comprising an elastic and to provide pressure on said piston to release a pulse of infusion liquid from said reservoir.

16. The device according to claim 8 wherein said piston further comprises a projection actuate a valve.

17. The device according to claim 8 wherein said reservoir and said piston are disposable.

18. A method for converting a constant flow of a fluid into a pulse flow, the method comprising the following steps:
releasing a fluid from an external reservoir, said fluid having a constant flow;
passing said fluid through a first one-way cheek valve;
containing a predefined volume of said fluid in an internal reservoir; and
releasing at least one pulse of fluid from said internal reservoir through a second one-way cheek valve.

19. The method of claim 18 wherein said second one-way cheek valve is a pressure-activated check valve.

20. The method according to claim 18 wherein said release at said fluid form said internal reservoir is in a plurality of consecutive pulses.
